(19) United States
(12) Patent Application Publication
(10) Pub. No.: US 2022/0068503 A1
(43) Pub. Date: Mar. 3, 2022

(54) Web-Based Personalized Health Management System

(71) Applicant: USARAD HOLDINGS, INC., Fort Lauderdale, FL (US)

(72) Inventor: Michael Yuz, Oakland Park, FL (US)

(73) Assignee: USARAD HOLDINGS, INC., Fort Lauderdale, FL (US)

(21) Appl. No.: 17/007,620

(22) Filed: Aug. 31, 2020

(51) Int. Cl. G16H 80/00; H04L 29/08; G16H 10/60; H04L 9/06

(52) U.S. Cl. CPC G16H 80/00; H04L 67/2809; H04L 2209/38; H04L 9/0637; G16H 10/60

(57) ABSTRACT

A method including receiving, at a processing device of a health management server, a request from a first remote computing device, the request indicating a request by a patient for a general second opinion and a personalized health plan from a physician, determining, at the processing device, one or more physicians who match the request, providing, from the processing device, a list of the one or more physicians to the first remote computing device, brokering, at the processing device, a consultation between patient and physician, receiving, at the processing device, the general medical second opinion from a second remote computing device, generating, at the processing device, the personalized health plan, providing, from the processing device, the personalized health plan to the first remote computing device, and receiving and processing electronic payment from the patient after receipt of the personalized health plan, the electronic payment being governed by blockchain technology.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, TABLE OR COMPUTER PROGRAM LISTING APPENDIX

Not Applicable

### BACKGROUND OF THE INVENTION

### 1. Field of the Invention

The present invention relates generally to systems and methods for managing patient health. In particular, examples of the present invention are related to a web-based personalized health management system for providing a general medical second opinion and a personalized health plan to a patient.

### 2. State of the Prior Art

Personalized medicine is a medical model that provides a customized approach to patient healthcare by taking into account information particular to a patient in determining medical decisions, practices and products. While patient healthcare has always been personal to the patient, personalized medicine, leveraging advances in digital technologies, human genetics, and molecular biology, provides a level of personalization not previously achievable by traditional medicine. For example, genetic testing for disease-causing mutations in certain genes can inform patients as to whether they are at higher risk for cancer and suggest individualized prophylactic therapies.

Integral to personalized medicine is the identification of a patient's immediate and long-term health risks to establish goals and actions to decrease risks and improve health. The goals and actions can form part of a personalized health plan that takes into account all available patient information such as the patient's medical history, genetic testing, and relevant laboratory test results.

Digital technologies can be utilized both prior to, and following, the development of the patient personalized health plan. For example, applications running on smart phones are capable of collecting and electronically transmitting accurate diagnostic information such as patient weight, blood pressure, laboratory test results and electrocardiograms. Digital diagnostic devices such as the hand-held mChip can perform intricate medical tests for illnesses such as sexually transmitted diseases and prostate cancer. These technologies provide healthcare professionals with personalized patient information that can form, together with other patient information, the basis for a patient personalized health plan. Relying upon digital devices, physicians can monitor patient progress toward established goals from a distance making inefficient, inconvenient and expensive physician office and hospital visits less necessary.

Further, digital technologies enable patients with access to the Internet to consult with medical professionals located miles or even continents away. For example, telemedicine services enable healthcare providers to remotely connect with patients. These services include consultations using video conferencing, digital examination devices and clear voice conversations. Telemedicine products using the latest medical technology, including examination cameras, peripherals, scopes, remote vital sign monitors and fully-equipped telemedicine carts can be used to enhance the consultation.

A medical second opinion is an opinion received from a consultation with a physician other than the one a patient originally consulted with in order to get an opinion from a differing point of view. The second opinion is often an important factor considered by a patient deciding on an ideal course of treatment and can form part of the patient's personalized health plan. The opinion of a second physician can help avoid problems such as misdiagnosis, as well as give the patient peace of mind in knowing that a recommended procedure is the best course of treatment and is agreed upon by more than one professional. A second opinion also empowers a patient to make informed decisions in selecting their health care provider and where they receive their health care.

There are many reasons for a patient to seek a medical second opinion. Medical second opinions can be sought by a patient under circumstances including: 1) when a physician recommends surgery; 2) when a physician diagnoses the patient with a serious illness (such as cancer) that the patient does not believe he has; 3) when a physician recommends a treatment for the patient other than what the patient believes is necessary; 4) when a physician recommends elective surgery and a second opinion is required by an insurance policy; 5) when the patient believes that they have a condition that a physician fails to diagnose; and 6) when a physician recommends the second opinion. Furthermore, the patient may seek a general medical second opinion that takes into account the patient's overall health condition.

The knowledge base of medical professionals has dramatically expanded in virtually all areas of expertise with the continuing development of new technologies, equipment and increases in computing power and Internet speed. This development has made increasing amounts of information accessible to medical professionals who in turn are accessible to patients by virtue of the digital computing devices used by such patients. In addition, keeping up with technological and scientific advances presents challenges to all physicians. Physicians who are subspecialists in their fields have emerged in response to these developments and challenges. For example, a physician who is also a radiologist may be considered a specialist, a bone radiologist may be considered a subspecialist, and a foot radiologist may be considered a super-specialist.

While many physicians are competent in their field of expertise, skills, training and opinions vary. Physicians have different personalities, different experiences and different training. Some physicians are more conservative and others are more aggressive. Further, experts, being human beings, make mistakes. Studies have reported that mistake rates for experts can be 30% and higher. A second opinion from another physician can provide a patient with insurance against these odds.

Depending upon the patient's location, availability and ability to travel and the accessibility to highly specialized experts, the patient may face barriers in getting a second opinion. Telemedicine allows patients to connect with the ideal physician for their condition, regardless of geographic restrictions. However, many medical doctors do not have access to telemedicine services and do not offer a convenient second opinion service.

While traditional medicine has focused primarily on the treatment of diseases after they have developed, personalized medicine provides a means by which a patient's health can be predicted and diseases prevented. As previously noted, advances in human genetics and molecular medicine enable healthcare providers to better understand the role of genetics in disease and to counsel their patients regarding specific therapies tailored to their specific conditions. This preventative approach provides a cost-effective and health-enhancing alternative to traditional medicine and is expected to reduce healthcare costs considerably.

### BRIEF SUMMARY OF INVENTION

In accordance with embodiments of the present disclosure, a web-based personalized health management system for providing a general medical second opinion and a personalized health plan to a patient provides for the virtualization of patient health management. Patient health management services including matching patients with physicians and providing communication, negotiation and consultation between patients and physicians are easily accessible via a transparent, secure, and on-demand web portal. The personalized health management system can provide a patient with a personalized health plan in a timely and cost-effective manner.

In accordance with embodiments of the present disclosure, the web-based personalized health management system can include a web-based portal that enables registered patients and physicians to communicate with each other and with a health management server over a network. The communication can include sending a request from a patient computing device to the health management server requesting a general medical second opinion from a physician. As used herein, the term “computing device” can refer to mobile computing devices, such as mobile telephones, laptop computers, and tablet computers, or stationary computing devices. The request can include patient answers to a medical history questionnaire. The health management server can perform a search based on the request and send a list of physicians that match the request to the patient computing device. The patient can select a physician from the list of physicians and send the selection to the health management server. The health management server can serve as a broker between the patient and the selected physician as the terms and conditions of a consultation between the patient and the selected physician are negotiated.

In some embodiments of the present disclosure, the communication between the patient computing device and the health management server can include sending a request from the patient computing device to the health management server requesting a personalized health plan. The health management server can generate the personalized health plan from the patient medical history questionnaire and the general medical second opinion developed by the physician following the consultation with the patient. The health management server can transmit the personalized health plan to the patient computing device.

In some embodiments, a method is disclosed that includes receiving, at a processing device of a health management server, a request from a first remote computing device, the request being indicative of a request by a patient for a general medical second opinion and a personalized health plan from a physician and including patient answers to a medical history questionnaire; determining, at the processing device, a list of one or more physicians who match the request; providing, from the processing device, the list of the one or more physicians to the first remote computing device; brokering, at the processing device, a consultation between the patient and the physician; receiving, at the processing device, the general medical second opinion from a second remote computing device; generating, at the processing device, the personalized health plan, the personalized health plan being generated from information in the general medical second opinion; providing, from the processing device, the personalized health plan to the first remote computing device; and receiving and processing electronic payment from the patient following the receipt of the personalized health plan by the patient, the electronic payment being governed by blockchain technology.

In other embodiments, A health management server is provided, the health management sever having a processing device. The health management server includes a search module configured to: receive a request from a first remote computing device, the request being indicative of a request by a patient for a general medical second opinion and a personalized health plan from a physician, the request including patient answers to a medical history questionnaire; determine a list of one or more physician that match the request; and provide the list of one or more physicians to the first remote computing device. The health management server also includes a health management module configured to broker a consultation between the patient and the physician; generate the personalized health plan from the general medical second opinion; and receive and process electronic payment from the patient following the receipt of the personalized health plan by the patient, the electronic payment being governed by blockchain technology.

### BRIEF DESCRIPTION OF THE DRAWING FIGURES

Non-limiting and non-exhaustive embodiments of the present disclosure are described with reference to the following figures, wherein like reference numerals refer to like parts throughout the various views unless otherwise specified.

FIGS. 1A-1I are schematics illustrating a patient computing device and a physician computing device displaying a graphical user interface in communication with a health management server in accordance with some embodiments of the present disclosure;

FIG. 2 is a block diagram illustrating components of the computing devices of FIGS. 1A-1G;

FIG. 3 is block diagram illustrating components of the health management server of FIGS. 1A-1I; and

FIG. 4 is a flow chart illustrating an example method for providing a general medical second opinion and a personalized health plan in accordance with some embodiments of the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help improve the understanding of various embodiments of the present disclosure. Also, common but well-known elements that are useful or necessary in a commercially feasible embodiment are often not depicted in order to facilitate a less obstructed view of these various embodiments of the present disclosure.

### DETAILED DESCRIPTION OF A PREFERRED EMBODIMENT

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

Reference throughout this specification to “one embodiment”, “an embodiment”, “one example”, or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Embodiments in accordance with the present invention may be embodied as an apparatus, method, or computer program product. Accordingly, the present invention may take the form of an entirely hardware embodiment, an entirely software embodiment (including firmware, resident software, micro-code, etc.), or an embodiment combining software and hardware aspects that may all generally be referred to herein as a “module” or “system.” Furthermore, the present invention may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present invention may be written in any combination of one or more programming languages.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

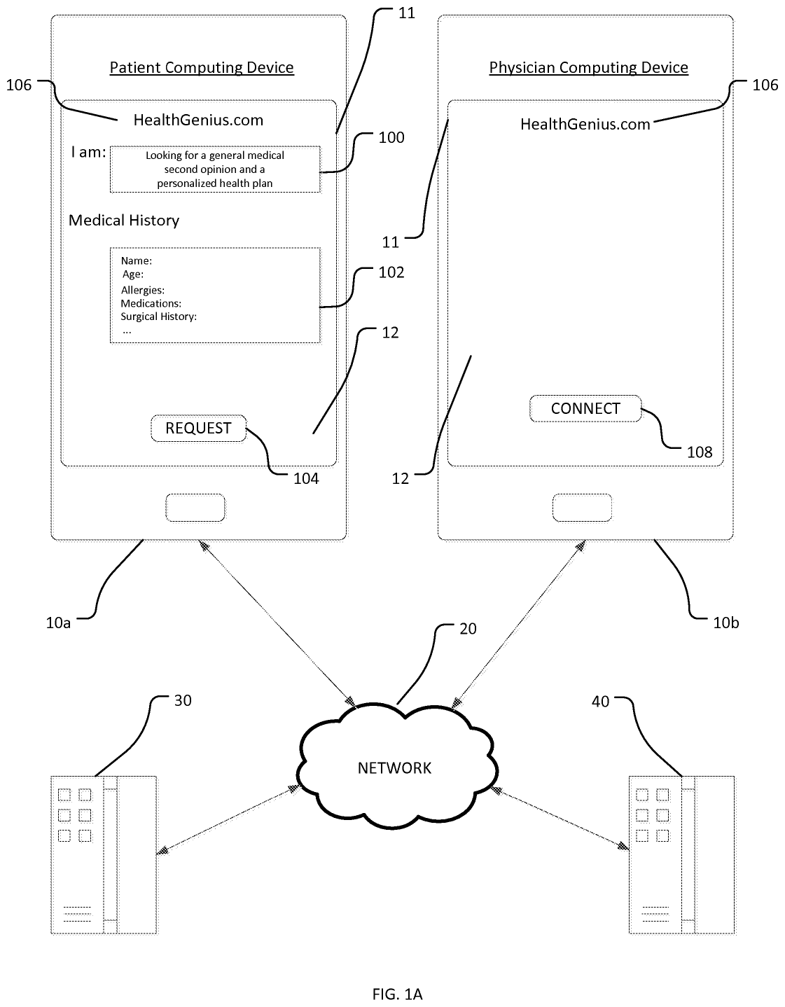

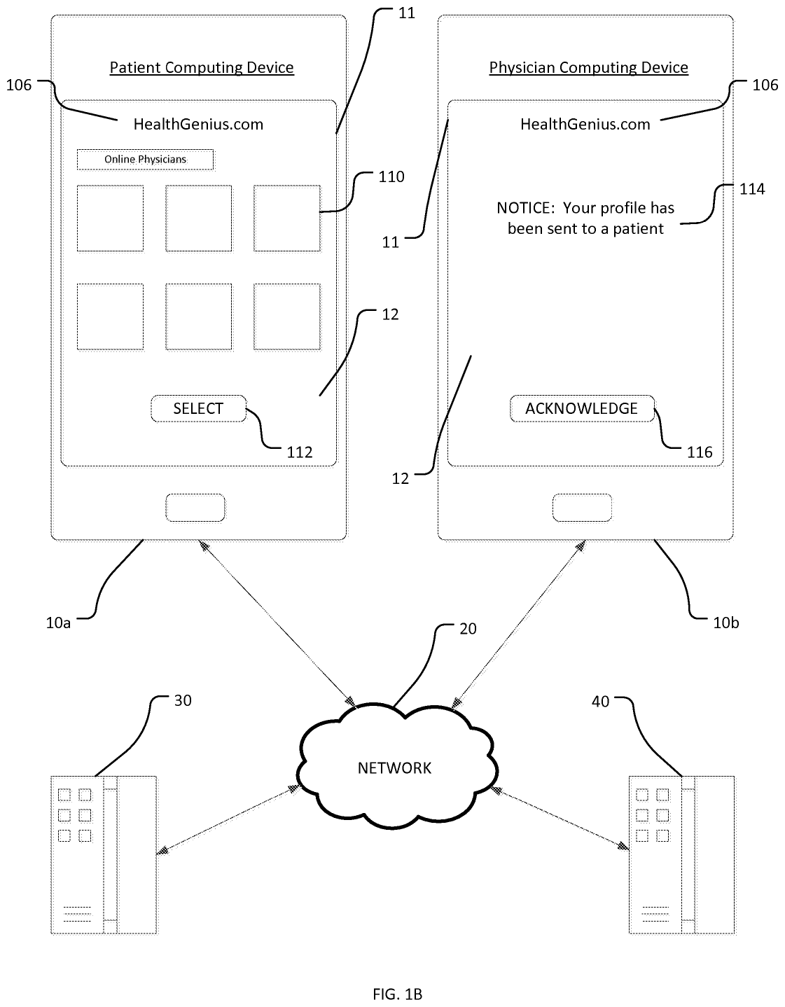

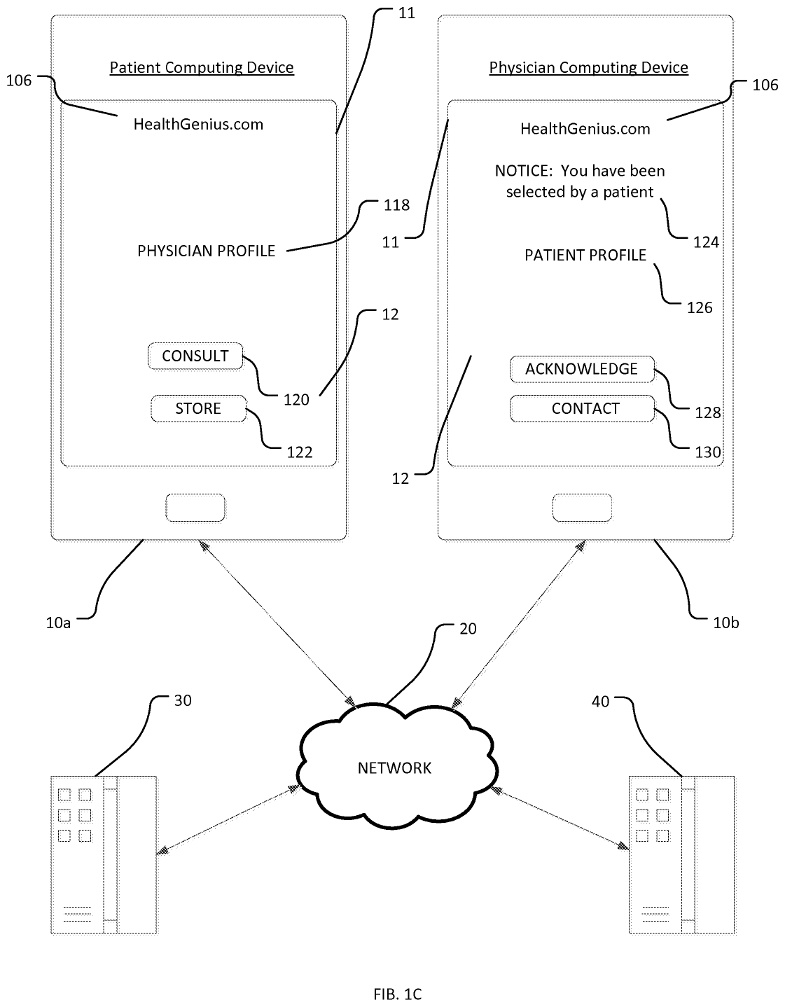

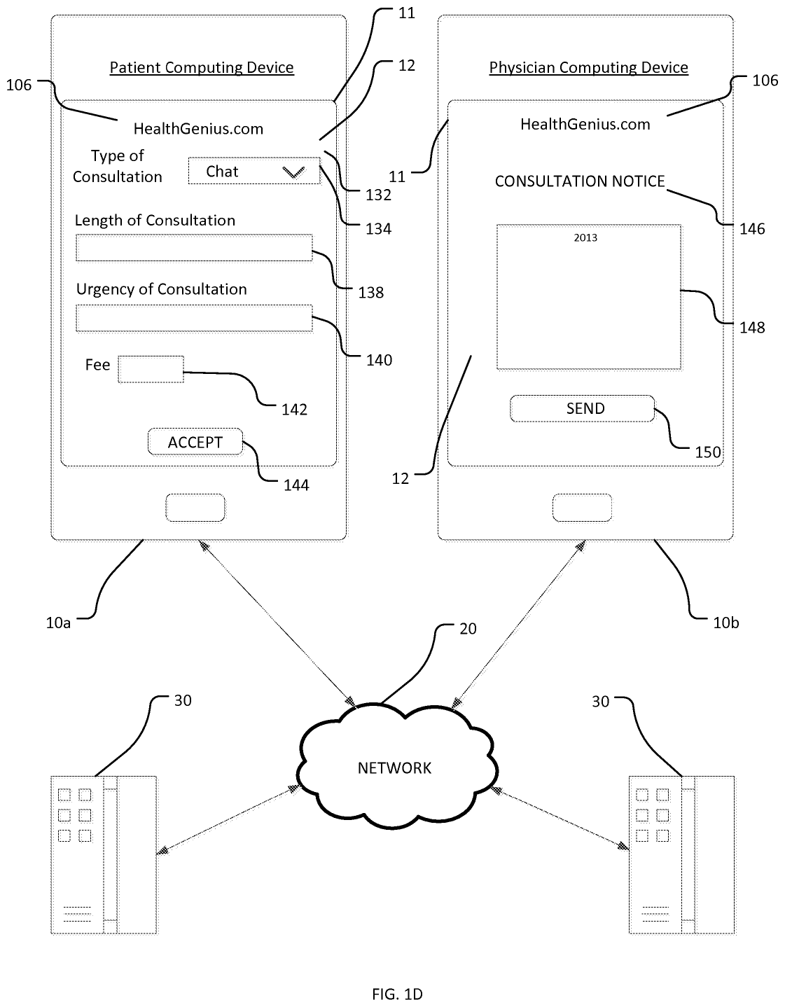

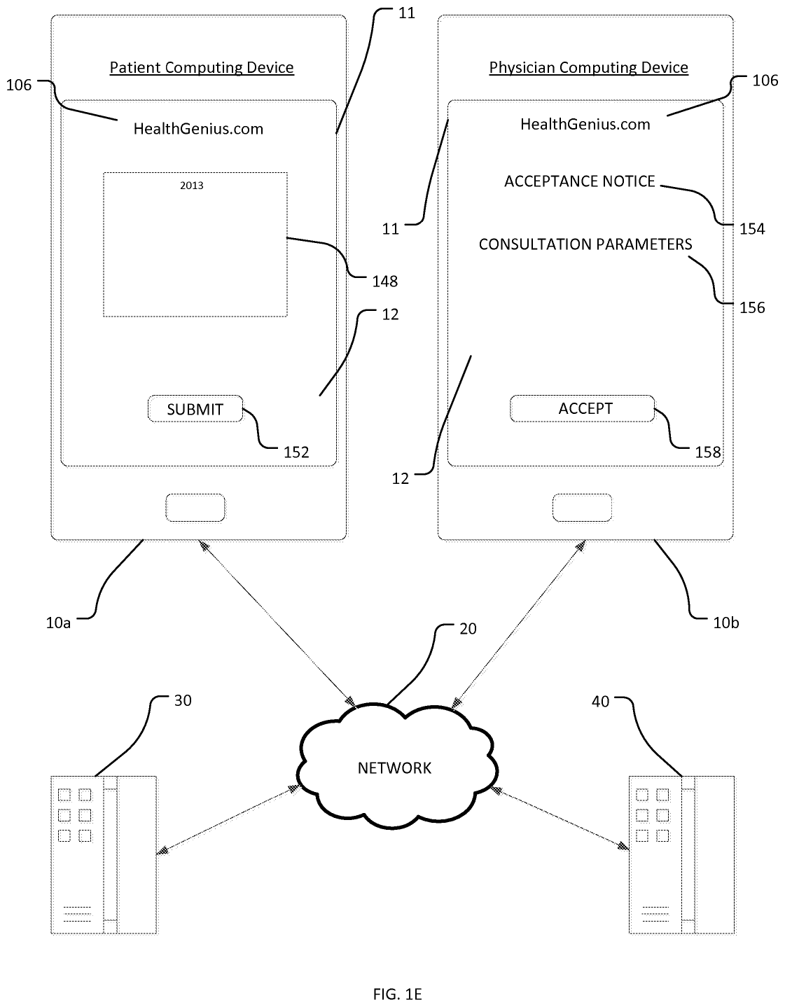

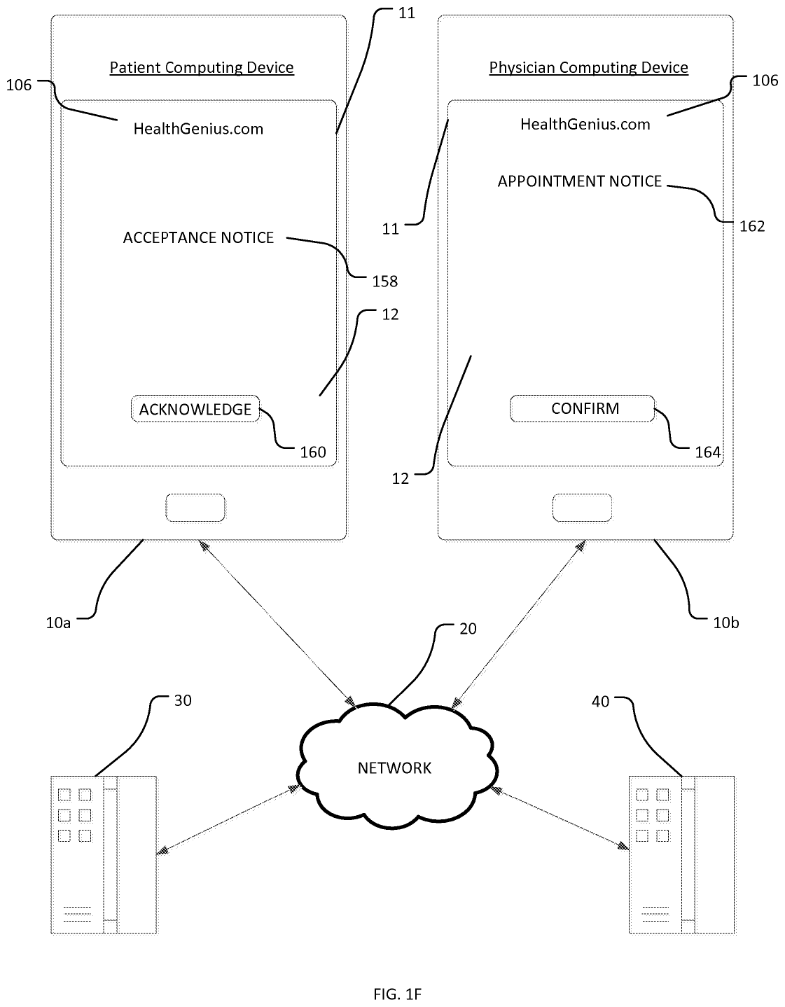

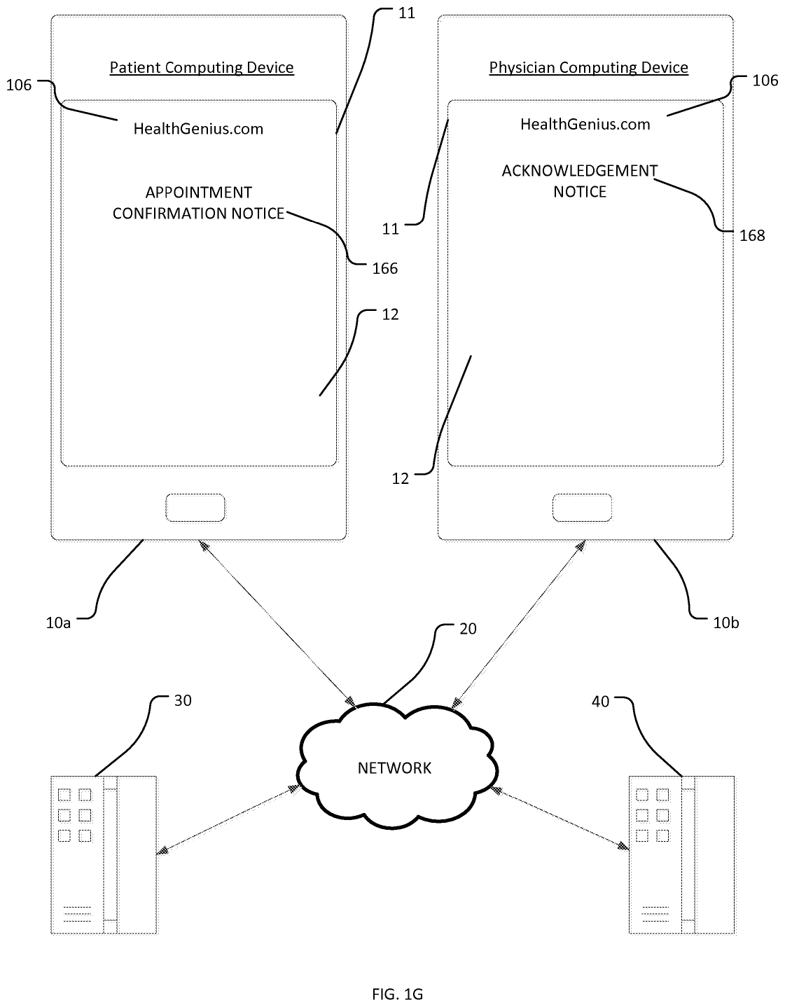

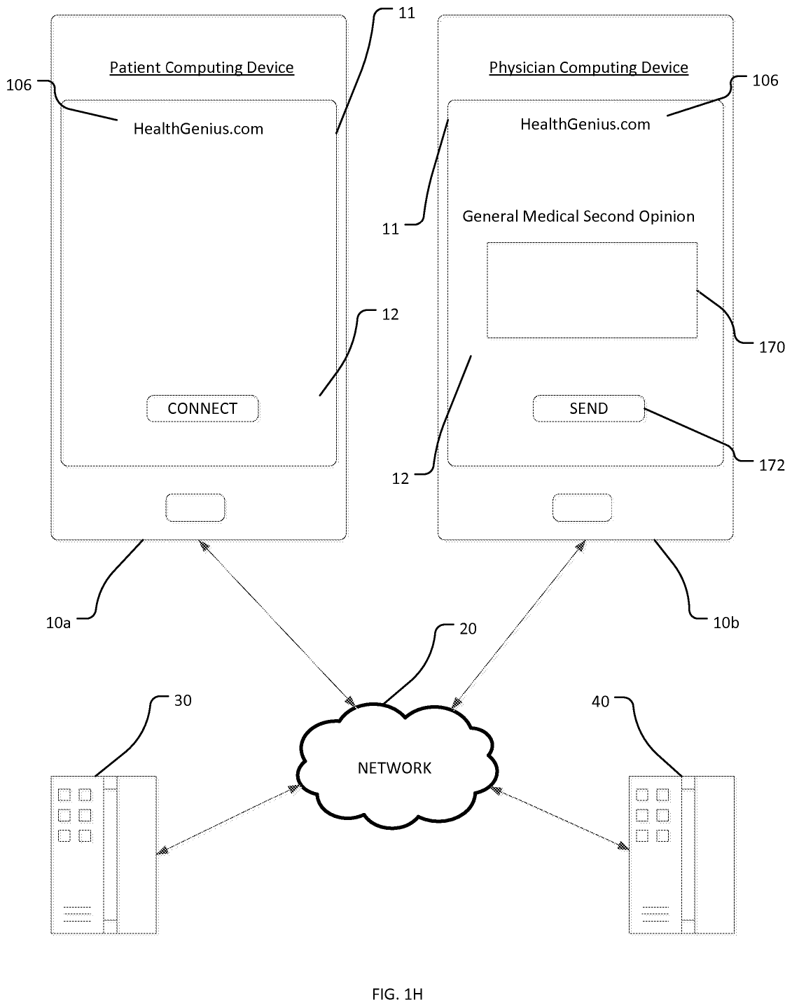

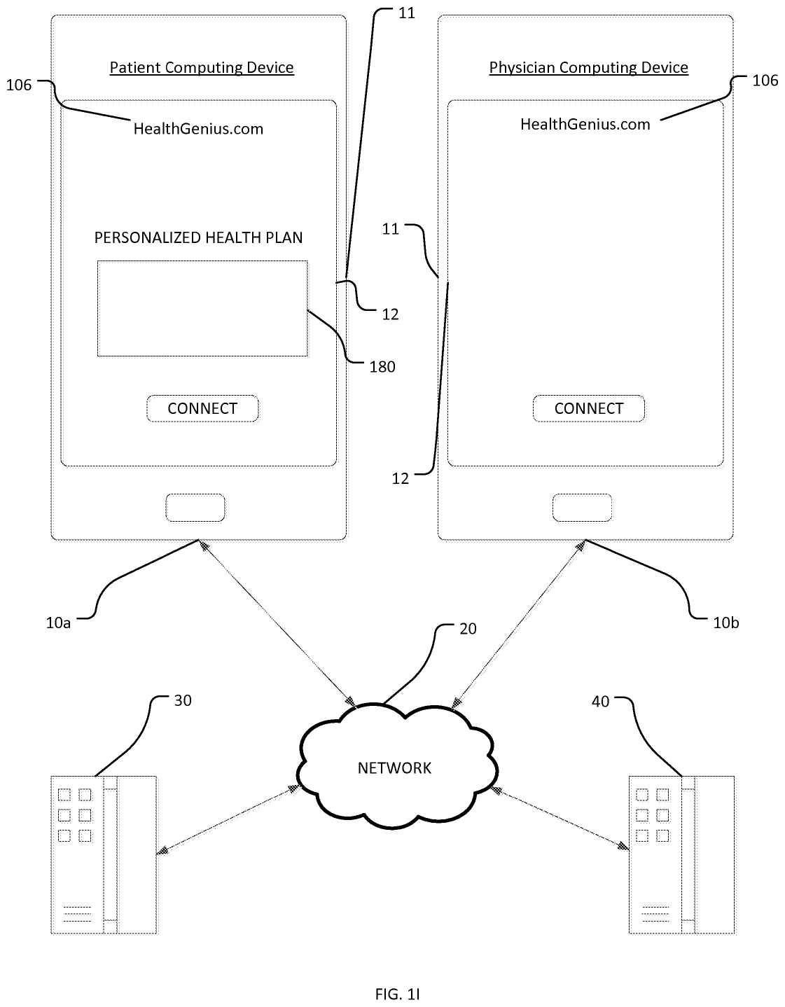

Referring now to FIG. 1A, computing devices 10a and 10b in communication with a health management server 30 via a network 20 are illustrated. Computing device 10a can be a computing device used by a patient while computing device 10b can be a computing device used by a physician. While one health management server 30 is illustrated, the term “health management server” refers to one or more servers that operate in an individual or distributed manner. Further, as used herein, the term “network” can refer to any communication network including, but not limited to, a wireless network, a cellular network, an intranet, the Internet, or combinations thereof. In the illustrated example, the computing devices 10a and 10b are displaying a graphical user interface (GUI) 12 on a touch screen 11 of the computing devices 10a and 10b. While a touch screen 11 is illustrated, it should be appreciated that other user interfaces can be used to allow the patient and the physician to interact with their respective computing devices 10a and 10b. For example, in some embodiments of the present disclosure the user interface may employ voice recognition techniques to accept patient and physician user input.

A third party server 40 is also illustrated. The third party server 40 can be a telemedicine or telehealth server. Operation of the third party server 40 is described in greater detail below.

In one embodiment, the GUI 12 of the computing device 10a allows the patient to send a request for a general second opinion and a personalized health plan to the health management server 30. From a HealthGenius.com application represented by the icon 106, the GUI 12 can display a menu 100 selection making explicit the patient's request. The GUI 12 can also display a field 102 that allows the patient to enter answers to a medical history questionnaire. The non-exhaustive medical history questionnaire illustrated includes exemplary questions such as the patient's name, age, allergies, medications being taken and surgical history. In some embodiments of the present disclosure, the field 102 allows the patient to enter specific health concerns such as hereditary cancer risk, body weight, diabetes/hypertension, skin issues, heart issues and back pain after surgery. In other embodiments of the present disclosure, the field 102 allows the patient to enter the results of lab tests, including testing for disease-causing mutations. In yet other embodiments of the present disclosure, the field 102 allows the patient to enter existing second opinions. The GUI 12 can further display a button 104 that indicates that the patient is requesting a general second opinion and a personalized health plan from a physician.

When the patient has entered his answers to the medical history questionnaire in field 102, he can press, or otherwise select, the button 104 to request a list of physicians available and qualified to provide a general medical second opinion. When the patient presses the button 104, the computing device 10a can transmit a request to provide a list of physicians to the health management server 30 via the network 20. The request can include the answers to the medical history questionnaire entered by the patient. In some embodiments of the present disclosure, the computing device 10a can present additional menus to the patient, such that the menus prompt the patient to select characteristics and qualifications of the physician he is seeking.

As illustrated, the GUI 12 of the computing device 10b can display the HealthGenius.com application icon 106 and a button 108 that upon being selected by the physician connects the physician with the health management server 30.

The health management server 30 can receive the request from the computing device 10a and can perform a request analysis to specify and formalize the patient's request and to determine a geographical location of the patient. In some embodiments of the present disclosure, the patient's request can be analyzed and represented by at least one keyword.

The health management server 30 can then query a database for physicians that match the patient's request. In some embodiments of the present disclosure, a physician is matched to the patient's request based on current availability and proximity to the patient. In other embodiments of the present disclosure, a physician is matched to the patient's request based on the presenting condition of the patient. In some other embodiments of the present disclosure, more than one physician can match the patient's request and a list of physicians can be generated by the health management sever 30. The health management server 30 transmits the list of physicians back to the computing device 10a.

Upon receiving the list of physicians from the health management server 30, the computing device 10a can display the list of physicians in the GUI 12, thereby allowing the patient to select a physician from the list of physicians. FIG. 1B illustrates an example of the computing device 10a displaying a list of physicians 110. In some embodiments of the present disclosure, the list of physicians 110 can include physicians that are currently online and include general information about each listed physician. In other embodiments of the present disclosure, the list of physicians can include a more detailed profile of each physician including their respective specialties and sub-specialties. The GUI 12 can further display a button 112 that indicates that the patient is selecting a physician from the list of physicians 110.

The health management server 30 can send a notice 114 to the computing device 10b that can be displayed by the GUI 12. The notice can notify the physician that his name and/or profile have been sent to a patient. The GUI 12 can further display a button 116 that indicates that the physician acknowledges receipt of the notice 114.

The health management server 30 can receive the patient's selection and transmit the selected physician's profile to the computing device 10a. FIG. 1C illustrates an example of the computing device 10a displaying the physician profile 118. The GUI 12 can further display a button 120 that indicates that the patient is requesting a consultation with the physician. The GUI 12 can also display a button 122 that indicates that the patient is selecting a physician's store from which the patient can purchase any of a plurality of items.

The health management server 30 can also transmit a notice and a patient profile to the computing device 10b. The notice can notify the physician that he has been selected by the patient. The GUI 12 can display the notice 124 and a patient profile 126. The patient profile 126 can include the patient's answers to the medical history questionnaire as well as other information such as the patient's preferred method of communicating with the physician. The GUI 12 can further display a button 128 that indicates that the physician acknowledges receipt of the notice 124 and the patient profile 126. The GUI 12 can also display a button 130 that indicates that the physician is contacting the patient.

The health management server 30 can receive the patient's consultation request and transmit a fee calculator to the computing device 10a. FIG. 1 D illustrates an example of the computing device 10a displaying the fee calculator 132. The fee calculator 132 can include a drop-down menu 134 to indicate a type of consultation preferred by the patient, a slider 138 to indicate the length of consultation desired by the patient, a slider 140 to indicate the urgency of the consultation, and a fee indicator 142. The type of consultation can include an online chat, a phone consultation and a video conference. In some embodiments of the present disclosure, the fee indicator indicates a fee based upon the consultation parameters selected by the patient in the drop-down menu 134 and the sliders 138 and 140. The GUI 12 can also display a button 144 that indicates that the patient accepts the physician's fee displayed in the fee indicator 142.

The health management server 30 can also transmit a notice and a physician calendar to the computing device 10b. The physician's calendar can be maintained by the health management server 30. The notice can notify the physician that the patient has requested a consultation. The GUI 12 can display the consultation notice 146 and the physician's calendar 148. In one embodiment, the physician's calendar 148 can show the physician's availability for a consultation. The GUI 12 can also display a button 150 that indicates that the physician is sending the physician calendar to the health management server 30.

The health management server 30 can receive the patient's acceptance of the physician's fee and the consultation parameters and transmit the physician's calendar to the computing device 10a. FIG. 1E illustrates an example of the computing device 10a displaying the physician's calendar 148. In some embodiments of the present disclosure, the patient can select a time for a consultation with the physician from the physician's available times. The GUI 12 can display a button 152 that indicates that the patient is submitting his selection of a date and time for the consultation with the physician.

The health management server 30 can also transmit a fee acceptance notice and the consultation parameters to the computing device 10b. The notice can notify the physician that the patient has accepted the physician's fee. The GUI 12 can display the acceptance notice 154 and the consultation parameters 156. The GUI 12 can also display a button 158 that indicates that the physician is sending his acceptance of the patient's consultation parameters 156 to the health management server 30.

The health management server 30 can receive the patient's date and time submission and transmit an acceptance notice indicating the physician's acceptance of the patient's consultation parameters 156 to the computing device 10a. FIG. 1 F illustrates an example of the computing device 10a displaying an acceptance notice 158. The GUI 12 can display a button 160 that indicates that the patient acknowledges receipt of the acceptance notice 158.

The health management server 30 can also receive the patient's submission of his selection of a date and time for the consultation with the physician and transmit an appointment notice to the computing device 10b. The appointment notice 162 can include the date and time selected by the patient. The GUI 12 can display a button 164 that indicates that the physician confirms the appointment at the selected date and time.

The health management server 30 can receive the physician's appointment confirmation and transmit an appointment confirmation notice to the computing device 10a. FIG. 1G illustrates an example of the computing device 10a displaying an appointment confirmation notice 166. The brokerage server 30 can also receive the patient's acknowledgement of receipt of the acceptance notice 158 and transmit an acknowledgement notice to the computing device 10b. The GUI 12 can display the acknowledgement notice 168 on the computing device 10b.

Following the consultation with the patient, the physician can enter his general medical second opinion in a field 170 displayed by the GUI 12 of the computing device 10b as illustrated in FIG. 1H. In some embodiments of the present disclosure, the field 170 can allow the physician to enter a narrative of the general medical second opinion. In other embodiments of the present disclosure, the field 170 provides sub-fields for entering specific physician recommendations and observations. The physician's general medical second opinion can be based upon the patient's answers to the medical history questionnaire, the consultation with the patient and other information available to the physician such as information from distance monitoring devices and laboratory tests. In some embodiments of the present disclosure, the general medical second opinion can include recommendations made by the physician to the patient for future appointments, treatments and monitoring. By way of example, the physician can recommend that the patient make an appointment with a radiologist if the patient complains of back pain following surgery. The GUI 12 can also display a button 172 that indicates that the physician is sending the general medical second opinion to the health management server 30.

The health management server 30 can receive the general medical second opinion and process the general medical second opinion to generate a personalized health plan for the patient. In some embodiments of the present disclosure, the health management server 30 can parse the general medical second opinion using computational linguistics algorithms to generate the personalized health plan. In other embodiments of the present disclosure, the health management server 30 can analyze the sub-fields of the general medical second opinion to generate the personalized health plan. The personalized health plan can include recommendations that address the patient's most pressing health risks including recommendations for future medical procedures, treatments and appointments, lifestyle changes and a health monitoring regime.

The health management server 30 can send the personalized health plan to the computing device 10a. As illustrated in FIG. 1I, the GUI 12 can display a personalized health plan 180 to the patient.

The examples of FIGS. 1A-1I are provided for example only and not intended to be limiting. It should be appreciated that variations of the GUI 12 are contemplated and are within the scope of the disclosure. Furthermore, while mobile computing devices are depicted, the computing devices 10a and 10b can be stationary computing devices as well.

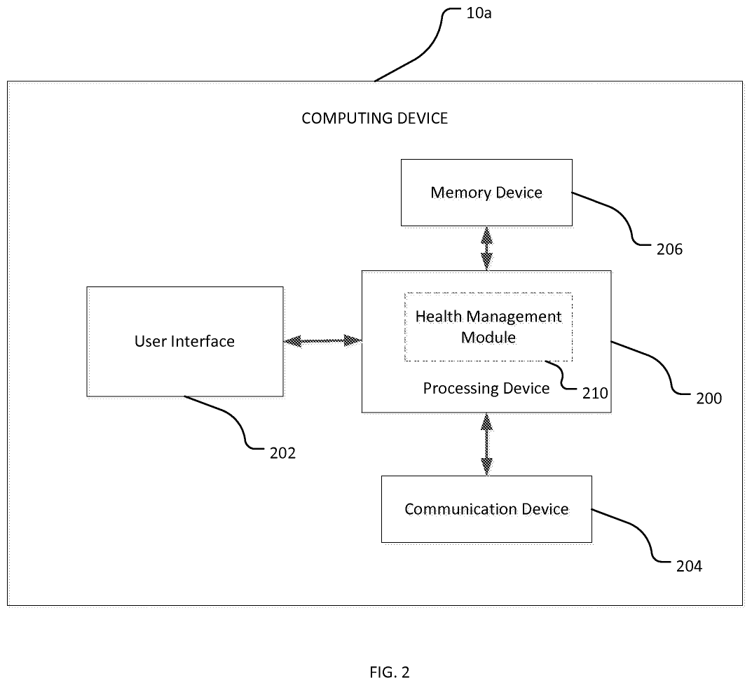

Referring now to FIG. 2, a block diagram illustrating example components of the computing devices 10a and 10b of FIGS. 1A-1I is depicted. Since the computing devices 10a and 10b are identical in all structural respects, the computing device 10a will be described. In the example embodiment, the computing device 10a includes a processing device 200, a user interface 202, a communication device 204, and a memory device 206.

The processing device 200 can include memory, e.g., read only memory (ROM) and random access memory (RAM), storing processor-executable instructions and one or more processors that execute the processor-executable instructions. In embodiments where the processing device 200 includes two or more processors, the processors can operate in a parallel or distributed manner. The processing device 200 can execute the operating system of the computing device 10a. In the illustrative embodiment, the processing device 200 also executes a health management module 210, which is described in greater detail below. The health management module 210 performs different functions dependent upon whether it is being executed by the computing device 10a or the computing device 10b.

The user interface 202 is a device that allows the patient to interact with the computing device 10a. While one user interface 202 is shown, the term “user interface” can include, but is not limited to, a touch screen, a physical keyboard, a mouse, a microphone, and/or a speaker. The communication device 204 is a device that allows the computing device 10a to communicate with another device, e.g., the health management server 30, via the network 20. The communication device 204 can include one or more wireless transceivers for performing wireless communication and/or one or more communication ports for performing wired communication. The memory device 206 is a device that stores data generated or received by the computing device 10a. The memory device 206 can include, but is not limited to a hard disc drive, an optical disc drive, and/or a flash memory drive.

In some embodiments, the health management module 210 is embodied as processor-executable instructions stored in the memory of the processing device 200. The processing device 200 can execute the health management module 210. The health management module 210 can be preloaded into the operating system of the computing device 10a, can be downloaded from a third party server by a user of the computing device 10a, or be generated on the computing device 10a.

The health management module 210 of the computing device 10a provides the GUI 12 and receives patient instructions via patient input. The health management module 210 further receives commands to request a general medical second opinion and a personalized health plan from the health management server 30. In response to receiving a list of physicians from the health management server 30, the health management module 210 displays the list of physicians in the GUI 12 and receives patient input indicating a selection of one of the physicians. In response to receiving a physician profile from the health management server 30, the health management module 210 displays the physician profile in the GUI 12 and receives patient input indicating a request for a consultation with the physician. In response to receiving a fee calculator from the health management server 30, the health management module 210 displays the fee calculator in the GUI 12 and receives patient input indicating consultation parameters such as the type of consultation desired (in person, phone, chat, video) and the length and urgency of the consultation. The health management module 210 further receives a physician's calendar from the health management sever 30, displays the physician's calendar in the GUI and receives patient input indicating a selection of a date and time for the consultation. It should be appreciated that the health management module 210 of the computing device 10a can be configured to perform additional functions without departing from the scope of the present disclosure.

The health management module 210 of the computing device 10b provides the GUI 12 and receives physician instructions via physician input. The health management module 210 further receives commands to acknowledge receipt of a patient profile from the health management server 30. In response to receiving a consultation request from the health management server 30, the health management module 210 displays a physician's calendar in the GUI 12 and receives physician input indicating the physician's intention to send the physician's calendar to the health management server 30. In response to receiving consultation parameters from the health management server 30, the health management module 210 displays the consultation parameters in the GUI 12 and receives physician input indicating the physician's acceptance of the consultation parameters. In response to receiving an appointment notice from the health management server 30, the health management module 210 displays the appointment notice in the GUI 12 and receives physician input indicating confirmation of the appointment. It should be appreciated that the health management module 210 of the computing device 10b can be configured to perform additional functions without departing from the scope of the present disclosure.

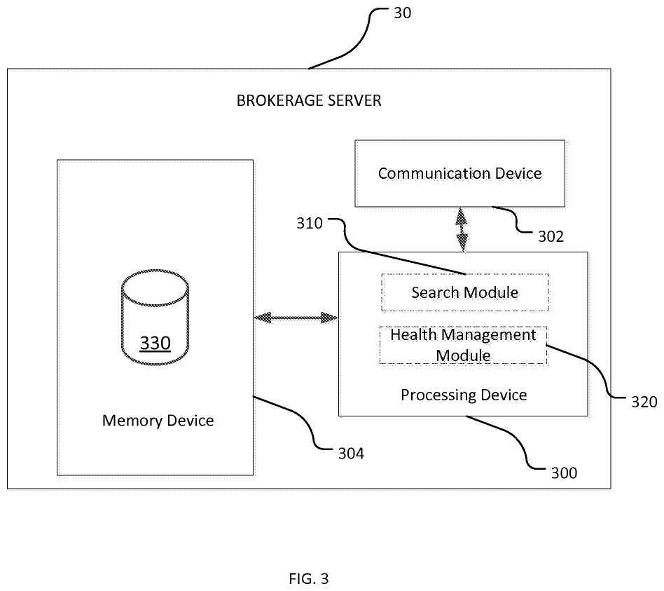

Referring now to FIG. 3, a block diagram illustrating an exemplary health management server 30 is depicted. In an exemplary embodiment, the health management server 30 includes a processing device 300, a communication device 302, and a memory device 304.

The processing device 300 can include memory, e.g., read only memory (ROM) and random access memory (RAM), storing processor-executable instructions and one or more processors that execute the processor-executable instructions. In embodiments where the processing device 300 includes two or more processors, the processors can operate in a parallel or distributed manner. In the illustrative embodiment, the processing device 300 specifically executes a search module 310 and a health management module 320, which are described in greater detail below. The processing device 300 is specifically configured to allow search module 310 and health management module 320 to perform the functions as described within this disclosure.

The communication device 302 is a device that allows the health management server 30 to communicate with another device, e.g., the computing devices 10a and 10b, via the network 20. The communication device 302 can include one or more wireless transceivers for performing wireless communication and/or one or more communication ports for performing wired communication.

The memory device 304 is a device that stores data generated or received by the health management server 20. The memory device 304 can include, but is not limited to, a hard disc drive, an optical disc drive, and/or a flash memory drive. The memory device 304 is accessible to the processing device 300. A participant database 330, or similar structures, can be stored in the memory device 304.

The participant database 330 stores information related to registered patients and physicians. Information stored in the participant database 330 can include patient data such as name, address, and date of birth as well as queries made by the patient. Information stored in the participant database 330 can also include physician data such as name, address, and date of birth, as well as the physician's calendar, areas of expertise and qualifications of the physician.

The search module 310 receives commands from the computing device 10a. The search module 310 receives a request for a general medical second opinion and a personalized health plan from the computing device 10a. As described, the request can include patient answers to a medical history questionnaire. The search module 310 performs a request analysis to specify and formalize the patient's request and to determine a geographical location of the patient. The patient's location can be determined using location-based technologies such as mobile GPS, HTMLS, and AJAX. In some embodiments of the present disclosure, the patient's request can be analyzed and represented by at least one keyword.

The search module 310 can query the participant database 320 and determine if there is a match between the patient's request and an available physician. The search module 310 can send a list of physicians matching the patient's request to the communication device 302 that in turn transmits the list of physicians to the computing device 10a.

The health management module 320 receives commands from the computing devices 10a and 10b. In response to receiving a patient's selection of a physician from the list of physician, the health management module 310 notifies the computing device 10b of the selected physician, provides the patient's profile to the computing device 10b, and provides the physician's profile to the computing device 10a. In some embodiments of the present disclosure, the patient and physician profiles can be stored in the participant database 320. The health management module 320 can also be configured to receive and process electronic payment from the patient following the receipt of the personalized health plan by the patient.

In one embodiment, the electronic payment is governed by blockchain technology. In one embodiment, the present disclosure utilizes blockchain technology to govern disputes, govern electronic payments, and establish communication directly between parties (i.e., patients and physicians) without third-party intermediaries. Blockchains are decentralized, peer-to-peer networked databases that can store and authenticate a ledger of assets and asset transactions. Blockchain technology allows transactions to occur without a third-party intermediary. Blockchain technology uses smart contracts to resolve disputes. Smart contracts are computer programs designed to act as self-executing contracts that implement aspects of a transaction where the terms of the agreement between buyer and seller are directly written into lines of software code. The code and the agreements exist across a distributed, decentralized blockchain network.

The present disclosure allows for transactions to take place across a blockchain database. For example, any electronic payment that is sent by the patient following the successful receipt of a general medical second opinion or a personalized health plan, can be governed by blockchain technology, specifically using a smart contracts concept, which allows for instantaneous payment.

Further, the present disclosure allows for all second opinions to be stored in a blockchain database. In one embodiment, processing device 300 automatically selects one or more physicians from a pool of physicians to render a second opinion. This selection can be based on physician criteria and expertise discussed herein.

In another embodiment, artificial intelligence (“AI”) algorithms are used to help physicians generate a general medical second opinion and/or personalized health plan faster and more accurately.

The health management module 320 receives a consultation notice from the computing device 10a and sends the computing device 10a a physician fee calculator. In some embodiments, the physician fee calculator can include patient-selectable fields indicating consultation parameters such as the type of consultation sought and the length and urgency of the consultation. The consultation parameters can be used to determine the physician's fee. In response to receiving a patient's acceptance of the fee determined by the physician fee calculator, the health management module 320 sends the acceptance notice and the consultation parameters to the computing device 10b and the physician's calendar to the computing device 10a in case the physician accepts the patient's consultation parameters. In response to receiving a patient's submission of a selected date and time for the consultation with the selected physician, the health management module 320 sends an appointment notice to the computing device 10b.

In some embodiments of the present disclosure, the consultation between the patient and the physician can be mediated by the health management module 320. For example, the health management module 320 can facilitate an online chat between the patient and the physician. In other embodiments, the consultation between the patient and the physician can be mediated by the health management module 320 and include the facilities of the third party server 40. For example, the third party server 40 can be a telemedicine server.

The health management module 320 can also receive the general medical second opinion from the computing device 10b and process the general medical second opinion to generate a personalized health plan. The health management server 30 can parse the general medical second opinion using computational linguistics algorithms to generate the personalized health plan. The health management server 30 can also analyze the sub-fields of the general medical second opinion to generate the personalized health plan. The health management module 320 can send the personalized health plan to the computing device 10a.

It is appreciated that the foregoing example of the health management server 30 is not intended to be limiting. Variations of the exemplary health management server 30 are contemplated and within the scope of the invention. For example, the memory device 304 may store a look-up table or a hash table that relates patient requests to potential physicians. In another example, the health management server 30 can utilize artificial intelligence techniques to associate keywords with each record in the participant database 330 to increase the robustness of the patient/physician matching process.

The health management server 30 can also include modules that provide administrative and security functions. For example, the processing device 300 can execute a registration module to provide patients and physicians with an interface for entering registration information such as name, address, date of birth, medical conditions, consultation preferences, and qualifications. Patient and physician relations modules can be executed by the processing device 300 to provide patients and physicians with an interface for managing their accounts. In other exemplary embodiments of the present disclosure, the processing device 300 can execute a coupon module that provides registered physicians with the ability to post promotions and coupons. In other exemplary embodiments of the present disclosure, the processing device 300 can execute a review module that provides registered patients and physicians with the ability to post reviews of a consultation and personalized health plan.

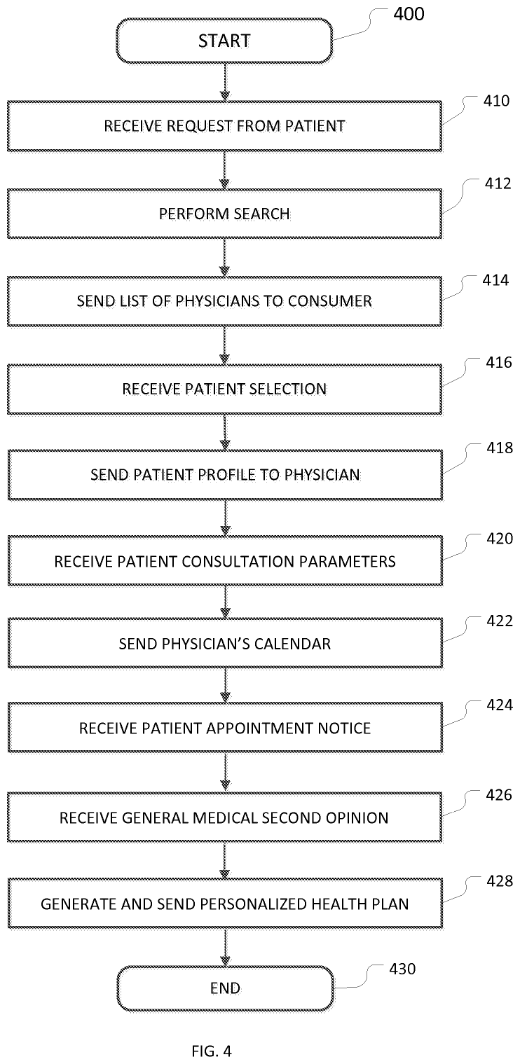

Referring now to FIG. 4, an example method 400 for automating the provision of a general medical second opinion and personalized health plan is illustrated. In some embodiments of the present disclosure, the health management server 30 (FIG. 3) executes the method 400. The flowchart and block diagrams in the flow diagrams illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

At operation 410, the search module 310 receives the request from the patient's computing device 10a. As discussed above, the GUI 12 of the patient's computing device 10a allows the patient to enter the request. At operation 412, the search module 310 conducts a search and determines if there is a match between the patient's request and a physician. At operation 414, the search module 310 provides the list of physicians to the patient's computing device 10a. In some embodiments of the present disclosure, the list of physicians can include a physician profile for each physician in the list.

At operation 416, the health management module 320 receives a patient selection indicating the patient's desire to consult with the selected physician. At operation 418, the health management module 320 sends the patient's profile to the physician's computing device 10b. As discussed above, the patient's profile can include the patient's answers to a medical history questionnaire. At operation 420, the health management module 320 receives consultation parameters from the patient's computing device 10a. As previously noted, the consultation parameters can include a physician fee. At operation 422, the health management module 320 sends the patient's computing device 10a the physician's calendar. The physician's calendar can show the patient the dates and times when the physician is available for a consultation. At operation 424, the health management module 320 receives the patient's appointment notice indicating the patient's selection of the date and time for the consultation. At operation 426, the health management module 320 receives the general medical second opinion from the computing device 10b. At operation 428, the health management module 320 generates and sends a personalized health plan to the computing device 10a.

The method 400 of FIG. 4 is provided for example and is not intended to be limiting. It should be appreciated that the method 400 can include additional operations and some operations may be varied without departing from the scope of the disclosure.

The present disclosure provides a web-based personalized health management system for providing a general medical second opinion and a personalized health plan to a patient. Virtualization of these services allows patients to easily consult with a physician who has available relevant patient medical information from which the personalized health plan is developed by the health management server. In some instances, the personalized health plan may result in an early diagnosis of disease. Patient conformance with the personalized health plan can be monitored using the described digital technologies to prevent the occurrence of disease and reduce associated healthcare costs.

The above description of illustrated examples of the present invention, including what is described in the Abstract, are not intended to be exhaustive or to be limitation to the precise forms disclosed. While specific embodiments of, and examples for, the invention are described herein for illustrative purposes, various equivalent modifications are possible without departing from the broader spirit and scope of the present invention.

I claim:

1. A computer-implemented method comprising the steps of:
receiving, at a processing device of a health management server, a request from a first remote computing device, the request being indicative of a request by a patient for a general medical second opinion and a personalized health plan from a physician and including patient answers to a medical history questionnaire;
determining, at the processing device, a list of one or more physicians who match the request;
providing, from the processing device, the list of the one or more physicians to the first remote computing device;
brokering, at the processing device, a consultation between the patient and the physician;
receiving, at the processing device, the general medical second opinion from a second remote computing device;
generating, at the processing device, the personalized health plan, the personalized health plan being generated from information in the general medical second opinion;
providing, from the processing device, the personalized health plan to the first remote computing device; and
receiving and processing electronic payment from the patient following the receipt of the personalized health plan by the patient, the electronic payment being governed by blockchain technology.

2. The computer-implemented method of claim 1, further comprising analyzing the request and representing the request as at least one keyword.

3. The computer-implemented method of claim 1, further comprising determining a geographical location of the first remote computing device.

4. The computer-implemented method of claim 1, wherein brokering the consultation between the patient and the physician further comprises:
receiving, at the processing device, a selection from the first remote computing device, the selection being indicative of a patient selection of a physician from the list; and
providing, from the processing device, a patient profile to the second remote computing device, the patient profile providing the physician with information related to the patient's request for the general medical second opinion.

5. The computer-implemented method of claim 4, wherein brokering the consultation between the patient and the physician further comprises:
receiving, at the processing device, patient consultation parameters from the first remote computing device;
providing, from the processing device, the patient consultation parameters to the second remote computing device; and
receiving, at the processing device, an acceptance from the second remote computing device, the acceptance being indicative of acceptance by the physician of the patient consultation parameters.

6. The computer-implemented method of claim 5, wherein brokering the consultation between the patient and the physician further comprises:
providing, from the processing device, a physician's calendar to the first remote computing device;
receiving, at the processing device, an appointment notice from the first remote computing device, the appointment notice being indicative of a patient selection of an appointment date and time with the physician; and
providing, from the processing device, the appointment notice to the second remote computing device.

7. The computer-implemented method of claim 6, wherein brokering the consultation between the consumer and an expert further comprises:
receiving, at the processing device, a confirmation from the second remote computing device, the confirmation being indicative of the physician's confirmation of the patient-selected appointment date and time; and
providing, from the processing device, an appointment confirmation notice to the first remote computing device.

8. The computer-implemented method of claim 1, wherein generating the personalized health plan comprises parsing the general medical second opinion.

9. The computer-implemented method of claim 1, wherein generating the personalized health plan comprises analyzing a plurality of general medical second opinion sub-fields.

10. A health management server having a processing device, the health management server comprising:
a search module configured to:
(a) receive a request from a first remote computing device, the request being indicative of a request by a patient for a general medical second opinion and a personalized health plan from a physician, the request including patient answers to a medical history questionnaire;
(b) determine a list of one or more physician that match the request; and
(c) provide the list of one or more physicians to the first remote computing device; and
a health management module configured to:
(a) broker a consultation between the patient and the physician;
(b) generate the personalized health plan from the general medical second opinion; and
(c) receive and process electronic payment from the patient following the receipt of the personalized health plan by the patient, the electronic payment being governed by blockchain technology.

11. The health management server of claim 10, wherein the health management module is further configured to receive a selection from the first remote computing device, the selection being indicative of a patient selection of a physician from the list, and to provide a patient profile to a second remote computing device, the patient profile providing the physician with information related to the patient's request for the general medical second opinion.

12. The health management server of claim 11, wherein the health management module is further configured to receive patient consultation parameters from the first remote computing device, provide the patient consultation parameters to the second remote computing device, and receive an acceptance from the second remote computing device, the acceptance being indicative of acceptance by the physician of the patient consultation parameters.

13. The health management server of claim 12, wherein the health management module is further configured to provide a physician's calendar to the first remote computing device, receive an appointment notice from the first remote computing device, the appointment notice being indicative of a patient selection of an appointment date and time with the physician, and provide the appointment notice to the second remote computing device.

14. The health management server of claim 13, wherein the health management module is further configured to receive a confirmation from the second remote computing device, the confirmation being indicative of the physician's confirmation of the patient-selected appointment date and time, and provide an appointment confirmation notice to the first remote computing device.

15. The health management server of claim 10, wherein the health management module is further configured to receive the general second medical opinion from a second remote computing device and to generate the personalized health plan from the general second medical opinion.

16. The health management server of claim 15, wherein generating the personalized health plan comprises parsing the general medical second opinion.

17. The health management server of claim 15, wherein generating the personalized health plan comprises analyzing a plurality of sub-fields of the general medical second opinion.
